(19) United States
(12) Patent Application Publication
(10) Pub. No.: US 2023/0210833 A1
(43) Pub. Date: Jul. 6, 2023

(54) USE OF IRAK4 INHIBITOR IN TREATMENT OF ACUTE LUNG INJURY ALI/ARDS

(71) Applicant: SHANGHAI LEADINGTAC PHARMACEUTICAL CO., LTD., Shanghai (CN)

(72) Inventor: Yan FENG, Shanghai (CN)

(21) Appl. No.: 17/996,734

(22) PCT Filed: Apr. 16, 2021
(86) PCT No.: PCT/CN2021/087924

§ 371 Date: Oct. 20, 2022

(30) Foreign Application Priority Data
Apr. 20, 2020 (CN) 202010328648.7

(51) Int. Cl. A61K 31/4439; A61P 11/00

(52) U.S. Cl. CPC A61K 31/4439; A61P 11/00

(57) ABSTRACT

A use of an IRAK4 small-molecule inhibitor in preparation of a drug for treating or preventing acute lung injury or acute respiratory distress syndrome and related diseases thereof. Experiments prove that the IRAK4 small molecule inhibitor can obviously reduce the generation of inflammatory factors and prevent infiltration of eosinophil, neutrophil and lymphocyte, has excellent prevention and treatment effects on LPS-induced acute lung injury or acute respiratory distress syndrome, and is expected to become a new generation of the drug for treating acute lung injury and acute respiratory distress syndrome

### TECHNICAL FIELD

The invention belongs to the field of medical use, and specifically relates to the use of IRAK4 inhibitor in the preparation of drugs for preventing or treating ALI/ARDS diseases and related diseases thereof.

### BACKGROUND ART

Acute lung injury (ALI)/acute respiratory distress syndrome (ARDS) is an acute progressive hypoxic respiratory failure caused by various intrapulmonary and extrapulmonary pathogenic factors other than cardiogenic. ALI/ARDS has the same pathophysiological changes, which are two stages of the same disease. ALI represents the early stage and the relatively mild stage, while ARDS represents the more serious stage in the later stage. A variety of risk factors can induce ALI/ARDS, which are mainly divided into intrapulmonary factors (direct factors): such as severe lung infection, gastric content inhalation, lung contusion, inhalation of toxic gases, drowning, oxygen poisoning, etc., and extrapulmonary factors (indirect factors): such as severe infection, severe non-chest trauma, acute severe pancreatitis, massive blood transfusion, extracorporeal circulation, diffuse intravascular coagulation, etc.

At present, studies have found that the pathogenesis of acute lung injury is relatively complex. Apart from the direct damage of some pathogenic factors to pulmonary capillaries and lung tissue, more are pulmonary capillary endothelial cells and alveolar epithelial cells damage caused by uncontrolled lung inflammatory response indirectly mediated by a variety of inflammatory cells (such as macrophages, neutrophils, lymphocytes, etc.) and their released inflammatory mediators and cytokines (such as tumor necrosis factor-α(TNF-α), interleukin interleukin -6(IL-6)), interleukin-8 (IL-8), etc.), the permeability alveolar and pulmonary capillary is increased, leading to pulmonary edema and hyaline membrane formation. Due to alveolar edema and alveolar collapse, severe ventilation/blood flow ratio imbalance is caused, especially intrapulmonary shunt is significantly increased, resulting in severe hypoxemia. Pulmonary vasospasm and pulmonary microthrombosis formation trigger pulmonary hypertension.

Under the action of toxin factors, mononuclear macrophages (AM) can secrete more than 100 kinds of cytokines or inflammatory transmitters, such as TNF-α, IL-1, IL-6, IL-8, etc. In addition, AM can also activate vascular endothelial cells (EC) to produce IL-1, IL-8 and PAF, and release oxygen free radicals, proteases and various cytokines. The above factors are chemotactic factors of granulocytes and directly or indirectly participate in lung injury. IL-8 makes granulocytes degranulate through strong chemotactic effect on granulocytes, produces oxygen free radicals, proteases and other transmitters; increases the penetration of granulocytes into endothelial cell layer; promotes granulocytes through direct or indirect ways enter the interstitial gap and inflammatory area. IL-1 can promote the production of superoxide anions, and can activate neutrophils, increase the levels of protein differentiation antigens CD11 and CD12 expressed on the cell surface, and stimulate lung capillary endothelial cells to increase their surface intercellular adhesion molecule 1 (TCAM 1), leading to the interaction between white blood cells and endothelial cells, promoting the aggregation and adhesion of neutrophils (PMN), and releasing lysosomal enzymes, elastase and a large number of reactive oxygen species and superoxide ions, this can cause damage to vascular endothelial cells and alveolar epithelial cells.

Neutrophil (PMN) activation is the main cause of lung endothelial cell damage. Under normal circumstances, the number of PMN in the lung interstitium is quite small. In the early stage of ALI caused by various reasons, lung cells can produce a variety of direct chemotactic PMN substances, such as platelet activating factor (PAF), tumor necrosis factor α(TNF-α), complement C5a, etc. The above chemotactic substances can activate PMN, making a large number of PMN migrate and “sequester” in the pulmonary circulation, it adheres to the surface of pulmonary capillaries and releases a series of harmful substances that damage endothelial cells, such as PMN elastase and collagenase, toxic oxygen products (oxygen free radicals), PAF, etc. In addition to direct adhesion and damage to endothelial cells, PMN can also directly enter the alveolar cavity, causing epithelial damage and alveolitis. The permeability increases after endothelial and epithelial injury, allowing protein-rich fluid to leak into the stroma and alveolar cavity.

TNF-α is the initiator of ALI, which can play a role by inducing the production of NO, endothelin, oxygen free radicals, polypeptide transmitters, lipid transmitters and adhesion molecules, etc. As a proinflammatory factor, neutrophils can be adsorbed and stayed in the damaged part of lung tissue, combined with EC, and then transferred to lung parenchyma. Studies have shown that PMN adhesion to pulmonary capillary EC is an important way to cause ALI. In addition, TNF-α can cause lung injury through the following ways: TNF-α binds to TNF receptors in lung tissue, impairs lysosomes, and enzyme leakage causes lung injury; TNF-α stimulates granulocyte adhesion, “respiratory burst” and secondary cell degranulation, releasing protease, PAF and oxygen free radicals; stimulating mononuclear macrophages to produce IL-1, interleukin-2 (IL-2), interleukin-6 (IL-6) and interleukin-8 (IL-8) to cause tissue damage; TNF-α directly acts on EC to damage it, resulting in increased capillary permeability and thrombosis formation.

At present, the main therapeutic drugs for ALI/ARDS include anti-inflammatory drugs (glucocorticoids), antioxidants (N-acetylcysteine and propylcysteine), anticoagulant drugs (aspirin), antifungal drugs (ketoconazole), etc. However, all drugs have certain limitations in clinical application or need further clinical verification. Therefore, up to now, there is still a lack of effective drugs for ALI/ARDS treatment.

Interleukin-1 receptor kinase 4(IRAK4) is a serine/threonine-specific protein kinase, which plays an important role in activating the immune system. It is a key factor downstream of interleukin (IL)-1β family receptor and Toll-like receptor (TLR) signaling pathway. After it binds to MyD88, it activates IRAK1 or IRAK2, thereby transmitting signals to the downstream, thereby activating NF-κB and MAPK signaling pathways, it causes the expression and secretion of various inflammatory cytokines and anti-apoptosis molecules (such as TNF-α, IL-1, IL-6, IL-8, etc.). CA-4948, BAY-1834845, BAY-1830839, BMS-986126, and PF-06650833 are currently several IRAK4 inhibitors that are in the clinical development stage. They inhibit the activity of IRAK4 kinase to inhibit the activity of various inflammatory cytokines in the downstream signaling pathway mediated by IRAK4. They are mainly used in the treatment of cancer, inflammatory diseases, lupus erythematosus, diffuse large B-cell lymphoma and other autoimmune diseases. At present, there is no report of IRAK4 inhibitor for the treatment of ALI/ARDS.

### SUMMARY OF THE INVENTION

It is an object of the present invention to provide a novel pharmaceutical use of IRAK4 inhibitors, in particular to the use of IRAK4 inhibitors in the preparation of a medicament for the prevention or treatment of acute lung injury or acute respiratory distress syndrome or a related condition thereof, and to provide a novel strategy for the prevention or treatment of acute lung injury or acute respiratory distress syndrome or a related condition thereof.

The invention provides the use of IRAK4 inhibitors in the preparation of drugs for the prevention or treatment of acute lung injury or acute respiratory distress syndrome.

The invention provides the use of IRAK4 inhibitors in the preparation of drugs for preventing or treating acute lung injury.

The invention provides the use of IRAK4 inhibitors in the preparation of drugs for the prevention or treatment of acute respiratory distress syndrome.

The invention provides the use of IRAK4 inhibitors in the preparation of drugs for the prevention or treatment of acute lung injury or acute respiratory distress syndrome-related diseases.

In the first aspect of the present invention, it provides a use of an IRAK4 inhibitor for the preparation of a drug,
the IRAK4 inhibitor is a compound of formula I;

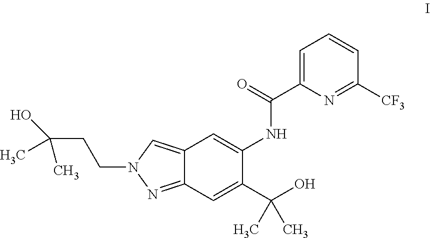 [SMILES: CC(C)(O)CCn1cc2cc(NC(=O)c3cccc(C(F)(F)F)n3)c(C(C)(C)O)cc2n1]

the drug is used to prevent and/or treat a disease selected from the group consisting of:
1) acute lung injury;
2) acute respiratory distress syndrome; and
3) acute lung injury or acute respiratory distress syndrome related disease.

In another preferred embodiment, the acute lung injury or the acute respiratory distress syndrome is caused by direct lung injury or indirect lung injury;
the direct lung injury is lung injury caused by lung contusion, aspiration, drowning, poison inhalation, diffuse lung infection, bacteria, virus, toxin or hypoxia;
the indirect lung injury is severe injury, shock, severe infection, severe non-chest trauma, sepsis, acute pancreatitis, massive blood transfusion, extracorporeal circulation, diffuse intravascular coagulation, or dermatomyositis.

In another preferred embodiment, the acute lung injury or the acute respiratory distress syndrome related disease is selected from the group consisting of non-cardiogenic pulmonary edema, acute hypoxic respiratory insufficiency or failure, hypoxemia, pulmonary interstitial fibrosis, pulmonary hypertension, and a combination thereof.

In another preferred embodiment, the disease is mediated by a cytokine selected from the group consisting of TNF-α, IL-6, IL-1β, and a combination thereof.

In another preferred embodiment, the disease is mediated by inflammatory cells selected from the group consisting of eosinophils, neutrophils, lymphocytes, and or combinations thereof.

In another preferred embodiment, the disease is an LPS-induced disease.

In another preferred embodiment, the drug is administered before LPS induction.

In another preferred embodiment, the drug is administered again after LPS induction.

In another preferred embodiment, the single dose of the drug is 100-200 mg/kg, preferably 135-165 mg/kg, more preferably 150 mg/kg.

In another preferred embodiment, the drug comprises:
1) a prophylactic and/or therapeutically effective amount of the compound of formula I; and
2) pharmaceutically acceptable carriers, diluents or excipients.

In another preferred embodiment, the drug further comprises other active ingredients for the prevention and/or treatment of acute lung injury or acute respiratory distress syndrome.

In another preferred embodiment, the dosage form of the drug is selected from the group consisting of tablets, pills, capsules, powders, granules, emulsions, suspensions, dispersions, solutions, syrups, elixirs, ointments, drops, suppositories, inhalants, and propellants.

In another preferred embodiment, the method of administration of the drug is selected from the group consisting of oral administration, sublingual administration, intravenous injection, intraperitoneal injection, intramuscular injection, subcutaneous injection, nasal administration, transdermal administration, parenteral administration, inhalation administration, intratracheal administration, intrapulmonary administration, bronchial administration, and a combination thereof.

In the second aspect of the present invention, it provides a drug for the prevention and/or treatment of acute lung injury or acute respiratory distress syndrome or related disease thereof, the drug comprises:
1) the compound of formula I as the first active ingredient;

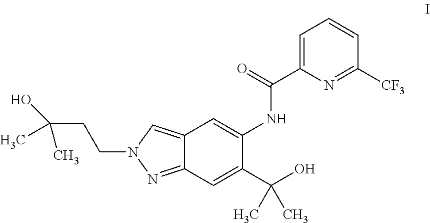 [SMILES: CC(C)(O)CCn1cc2cc(NC(=O)c3cccc(C(F)(F)F)n3)c(C(C)(C)O)cc2n1]

2) an optional second active ingredient, the second active ingredient is different from the first active ingredient, and the second active ingredient is used to prevent and/or treat acute lung injury or acute respiratory distress syndrome or related disease thereof;
3) pharmaceutically acceptable carriers, diluents or excipients.

In the present invention, the structural formula of compound 41 is

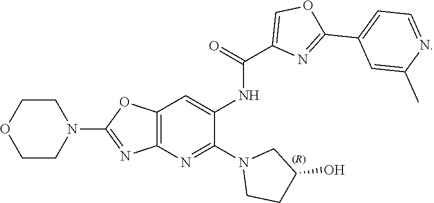

In the present invention, the IRAK4 inhibitor has the following structure:

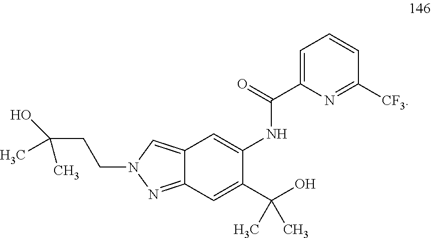 [SMILES: CC(C)(O)CCn1cc2cc(NC(=O)c3cccc(C(F)(F)F)n3)c(C(C)(C)O)cc2n1]

In the present invention, the structural formula of compound 450 is

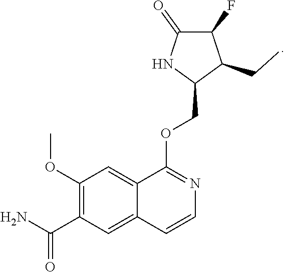 [SMILES: CC[C@@H]1[C@H](F)C(=O)N[C@@H]1COc1nccc2cc(C(N)=O)c(OC)cc12]

The present invention also provides a method of preventing or treating acute lung injury or acute respiratory distress syndrome, comprising administering to a patient in need thereof a therapeutically effective amount of an IRAK4 inhibitor.

The present invention also provides a method of preventing or treating acute lung injury comprising administering to a patient in need thereof a therapeutically effective amount of an IRAK4 inhibitor.

The present invention also provides a method of preventing or treating acute respiratory distress syndrome, comprising administering to a patient in need thereof a therapeutically effective amount of an IRAK4 inhibitor.

The present invention also provides a method of preventing or treating acute lung injury or acute respiratory distress syndrome related diseases comprising administering to a patient in need thereof a therapeutically effective amount of an IRAK4 inhibitor.

In the present invention, the acute lung injury or acute respiratory distress syndrome related diseases include, but are not limited to, non-cardiogenic pulmonary edema, acute hypoxic respiratory insufficiency or failure, hypoxemia, pulmonary interstitial fibrosis, pulmonary hypertension.

In the present invention, the acute lung injury or acute respiratory distress syndrome is caused by direct lung injury, including but not limited to lung contusion, aspiration, drowning, poison inhalation, diffuse lung infection, etc., or lung injury caused by bacteria, viruses, toxins, hypoxia.

In the present invention, the acute lung injury or acute respiratory distress syndrome is caused by indirect lung injury, including but not limited to sepsis, acute pancreatitis, severe extrapulmonary injury, shock, severe infection, severe non-chest trauma, acute severe pancreatitis, massive blood transfusion, extracorporeal circulation, diffuse intravascular coagulation, etc.

In the present invention, the IRAK4 inhibitor can effectively inhibit the activity of IRAK4 kinase, so as to achieve the effect of preventing or treating acute lung injury or acute respiratory distress syndrome or its related diseases.

In the present invention, the IRAK4 inhibitor can effectively regulate the level of inflammatory cells and/or inflammatory factors, so as to achieve the effect of preventing or treating acute lung injury or acute respiratory distress syndrome or its related diseases, wherein the inflammatory cells include but not limited to macrophages, neutrophils, lymphocytes, the inflammatory cytokines include, but are not limited to, TNF-α, IL-1β, IL-8, IL-6.

In the present invention, the IRAK4 inhibitor can effectively reduce the inflammatory reaction, so as to achieve the effect of preventing or treating acute lung injury or acute respiratory distress syndrome or its related diseases.

In the present invention, the drug comprises an IRAK4 inhibitor as an active ingredient; the drug of the present invention may also optionally comprise a pharmaceutically acceptable carrier, diluent or excipient.

In the present invention, the IRAK4 inhibitor can be used alone or combined with other drugs to achieve a better therapeutic effect on acute lung injury or acute respiratory distress syndrome.

In the present invention, the dosage form of the drug may be a tablet, a pill, a capsule, a powder, a granule, an emulsion, a suspension, a dispersion, a solution, a syrup, an elixir, an ointment, a drop, a suppository, an inhalant, and a propellant. The drug wherein IRAK4 inhibitor is an active ingredient thereof can be prepared into any of the above-mentioned drug dosage forms according to actual needs, each dosage form of the drug can be prepared in accordance with conventional methods in the pharmaceutical field.

In the present invention, according to actual needs, the drug administration route can be any one selected from oral administration, sublingual administration, intravenous injection, intraperitoneal injection, intramuscular injection, subcutaneous injection, nasal administration, transdermal administration, parenteral administration, inhalation administration, intratracheal administration, intrapulmonary administration, and bronchial administration.

The dosage of the compound of the present invention may be about 0.05-500 mg/kg body weight/day, such as 0.1-250 mg/kg body weight/day, 0.1-150 mg/kg body weight/day, 0.5-150mg/kg body weight/day.

Through experiments, the present invention proves that the IRAK4 inhibitor can significantly reduce the production of inflammatory factors and prevent the infiltration of eosinophils, neutrophils, and lymphocytes, and can significantly inhibit the pathological changes of lung tissue of ALI/ARDS mice, indicating that the IRAK4 inhibitor can be used for the treatment of ALI/ARDS and related diseases thereof.

So far, the clinically commonly used hormone drugs for the treatment of ALI/ARDS have side effects such as femoral osteonecrosis, mental disorders, and diffuse pulmonary interstitial fibrosis, but so far there is no better drug treatment method, and IRAK4 inhibitors can achieve the same or even better effects of hormone drugs and can avoid the above side effects.

The term “pharmaceutically acceptable salt” refers to an acid addition salt or a base addition salt of a compound of the present invention that is relatively non-toxic. The acid addition salt is a salt formed by a compound of formula (I) of the present invention and suitable inorganic or organic acid, these salts can be prepared during the final separation and purification of the compounds, or can be prepared by reacting the purified compounds of formula (I) in their free base form with suitable organic or inorganic acids. Representative acid addition salts include hydrobromate, hydrochloride, sulfate, bisulfate, sulfite, acetate, oxalate, valerate, oleate, palmitate, stearate, lauroleate, borate, benzoate, lactate, phosphate, hydrophosphate, carbonate, bicarbonate, toluate, citrate, maleate, fumarate, succinate, tartrate, benzoate, mesylate, p-toluene sulfonate, gluconate, lactobionate and lauryl sulfonate, etc. The base addition salt is a salt formed by a compound of formula (I) and a suitable inorganic or organic base, including, for example, a salt formed with alkali metals, alkaline earth metals, and quaternary ammonium cations, such as sodium salt, lithium salt, potassium salt, calcium salt, magnesium salt, tetramethylquaternary ammonium salt, tetraethyl quaternary ammonium salt, etc.; amine salt includes salts formed with ammonia (NH3), primary amine, secondary amine or tertiary amine, such as methylamine salt, dimethylamine salt, trimethylamine salt, triethylamine salt, ethylamine salt, etc.

In the present invention, the compound of formula I is compound 146.

### DESCRIPTION OF FIGURES

FIG. 1 is an HE staining light microscope of lung tissue of normal mice with a magnification of 400×.

FIG. 2 is an HE staining light microscope of lung tissue of LPS-induced ALI/ARDS model mice with a magnification of 400×.

FIG. 3 is an HE staining light microscope of lung tissue of LPS-induced ALI/ARDS model mice treated with compound 41 with a magnification of 400×.

FIG. 4 is an HE staining light microscope of lung tissue of LPS-induced ALI/ARDS model mice treated with compound 146 with a magnification of 400×.

FIG. 5 is an HE staining light microscope of lung tissue of LPS-induced ALI/ARDS model mice treated with compound 450 with a magnification of 400×.

### DETAILED DESCRIPTION OF THE INVENTION

After long-term and in-depth research, the present inventors have obtained a compound that has excellent preventive and/or therapeutic effects on ALI/ARDS diseases and related diseases thereof through extensive screening. Specifically, among the numerous IRAK4 inhibitors, the present inventors found that the compound 146 has significantly excellent inhibitory effects on TNF-α, IL-6 and IL-1β, has significantly reduced total cell count, eosinophil count, neutrophil count and lymphocyte count, and has significantly improved lung pathology score. On this basis, the inventors have completed the present invention.

The present invention will be described below in the form of specific embodiments. It should be understood that these specific embodiments are merely illustrative and not restrictive. Appropriate modifications and variations may be made to the present invention without departing from the spirit and scope of the invention. These modifications and variations are within the scope of the present invention. Various reagents used in the experiment can be purchased from the market, and their amounts are used in conventional quantities unless otherwise specified.

It should be understood that the compounds 146, 41 and 450 of the present invention can be prepared by the following preparation methods, can also be prepared by methods known in the art, or can also be commercially available.

### I COMPOUNDS PREPARATION EXAMPLES

### Example 1. Preparation of

N-[2-(3-hydroxy-3-methylbutyl)-6-(2-hydroxyprop-2-yl)-2H-indazol-5-yl]-6-(trifluoromethyl) pyridine-2-amide (146) (Refer to the Preparation Method of Example 11 in Patent CN107406416)

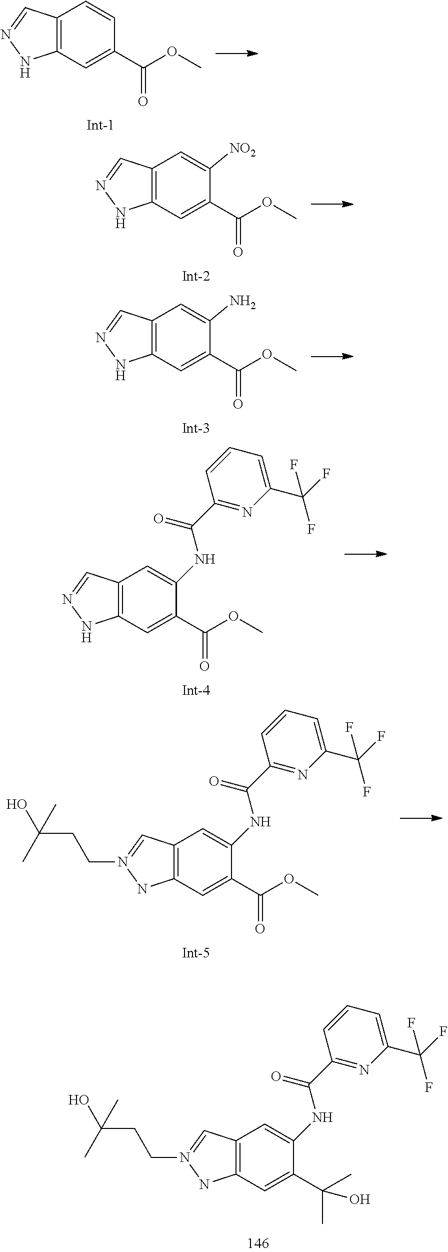

### Step 1: Preparation of methyl 5-nitro-1H-indazole-6 carboxylate (Int-2)

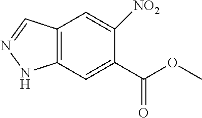 [SMILES: COC(=O)c1cc2[nH]ncc2cc1[N+](=O)[O-]]

In a three-necked flask, methyl 1H-indazole-6-carboxylate (4.60 g, 26.1 mmol) was dissolved in 120 ml of sulfuric acid (96%), the mixture was cooled to minus 15° C., cooled nitrating acid (10 ml of 96% sulfuric acid in 5 ml of 65% nitric acid) was added dropwise to the solution within 15 min. After completion of addition, the reaction solution was continued to be stirred for 1 h (internal temperature of −13° C.). The reaction solution was added to the ice, the precipitate was filtered out, washed with water, and dried at a low temperature of 50° C. in a drying oven. 5.10 g of target compound was obtained.

MS(ESI):m/z=222.1[M+H]⁺.

### Step 2: Preparation of methyl 5-amino-1H-indazole-6-carboxylate (Int-3)

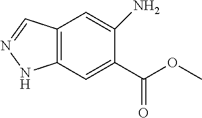

Methyl 5-nitro-1H-indazole-6-carboxylate (4.40 g, 19.8 mmol) was dissolved in 236 ml of methanol, hydrogenated with active palladium/carbon (1.06 g, 0.99 mmol) at 25° C. for 3 h under standard hydrogen pressure, the reaction solution was filtered over diatomite, washed with methanol, the filtrate was collected and concentrated to obtain 3.2 g of target compound.

### Step 3: Preparation of methyl 5-({[6-(trifluoromethyl) pyridine-2-yl] carbonyl}amino)-1H-indazole-6-carboxylate (Int-4)

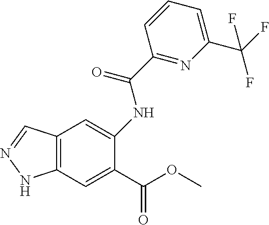 [SMILES: COC(=O)c1cc2[nH]ncc2cc1NC(=O)c1cccc(C(F)(F)F)n1]

6-(Trifluoromethyl) pyridine-2-carboxylic acid (3.3 g, 17.2 mmol) was added to 30 ml of tetrahydrofuran, followed by O-(benzotriazol-1-yl)-N,N,N,N-tetramethylurea tetrafluoroborate (6.05 g, 18.8 mmol) and N-ethyl-N-isopropyl propyl-2-amine (3.28 g, 18.8 mmol), and the reaction solution was stirred at room temperature for 30 min. Subsequently, methyl 5-amino-1H-indazole-6 carboxylate (3.0 g, 15.7 mmol) was added, and the reaction solution was stirred overnight at room temperature. The reactants were filtered through a membrane filter, the solids were washed with tetrahydrofuran and water, and the solids were dried overnight in a drying oven. 5g of target compound was obtained.

MS(ESI):m/z=365.2[M+H]⁺.

### Step 4: Preparation of methyl 2-(3-hydroxy-3-methylbutyl)-5-({[6-(trifluoromethyl) pyridine-2-yl] carbonyl}amino)-2H-indazole-6-carboxylate (Int-5)

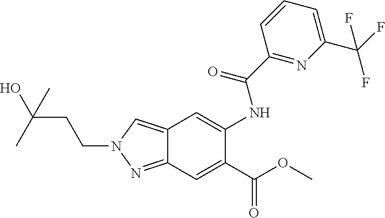 [SMILES: COC(=O)c1cc2nn(CCC(C)(C)O)cc2cc1NC(=O)c1cccc(C(F)(F)F)n1]

Methyl 5-({[6-(trifluoromethyl) pyridine-2-yl] carbonyl} amino)-1H-indazole-6-carboxylate (4.65 g, 12.75 mmol), potassium carbonate (5.3 g, 38.4 mmol) and potassium iodide (1.06 g, 6.4 mmol) were added to 45 ml of N,N-dimethylformamide, the mixture was stirred for 15 minutes, followed by 3.1 ml of 4-bromo-2-methylbuta-2-ol, and the reaction solution was stirred at 60° C. for reaction overnight. The reaction solution was washed with water and extracted three times with ethyl acetate, and the extract was washed three times with saturated sodium chloride solution. The extract was collected and concentrated. 2.5 g of target compound was obtained by column purification.

MS(ESI):m/z=451.2[M+H]⁺.

### Step 5: Preparation of N-[2-(3-hydroxy-3-methylbutyl)-6-(2-hydroxyprop -2-yl)-2H-indazole-5-yl]-6-(trifluoromethyl) pyridine-2-amide (146)

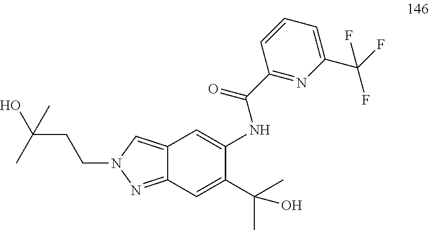 [SMILES: CC(C)(O)CCn1cc2cc(NC(=O)c3cccc(C(F)(F)F)n3)c(C(C)(C)O)cc2n1]

Methyl 2-(3-hydroxy-3-methylbutyl)-5-({[6-(trifluoromethyl) pyridine-2-yl] carbonyl} amino)-2H-indazole-6-carboxylate (2.1 g, 4.68 mmol) was added to 30 ml tetrahydrofuran, and the mixture was cooled in an ice-water bath. Then 3M methyl magnesium bromide ether solution (7.8 ml, 5.0 equivalent) was added, the reaction solution was cooled in an ice water bath while stirring for 1 h, and the reaction was stirred for 4.5 h at room temperature, then 1 equivalent methyl magnesium bromide solution was added, stirring for 21 h at room temperature, 1 equivalent methyl magnesium bromide solution was added again, and the reaction solution was stirred for 22 h at room temperature. The reaction solution was mixed with saturated ammonium chloride aqueous solution, stirred and extracted three times with ethyl acetate. The ethyl acetate phase was combined and washed with saturated sodium chloride solution, the organic phase was combined and concentrated. 2 g of target compound crude product was obtained. 750 mg of target compound was obtained by preparative HPLC purification.

MS(ESI):m/z=451.3[M+H]⁺.

¹H NMR (400 MHz, DMSO) δ12.36 (s, 1H), 8.71 (s, 1H), 8.45 (d, J=7.6 Hz, 1H), 8.41-8.32 (m, 2H), 8.16 (dd, J=7.7, 0.9 Hz, 1H), 7.57 (s, 1H), 5.95 (s, 1H), 4.51 (s, 1H), 4.50-4.43 (m, 2H), 2.10-1.96 (m, 2H), 1.62 (s, 6H), 1.15 (s, 6H).

### EXAMPLE 2. Preparation of (R)-N-(5-(3-hydroxypyrrolidine-1-yl)-2-morpholinoxazo [4,5-b] pyridine-6-yl)-2-(2-methylpyridine-4-yl) oxazole-4-formamide (41)

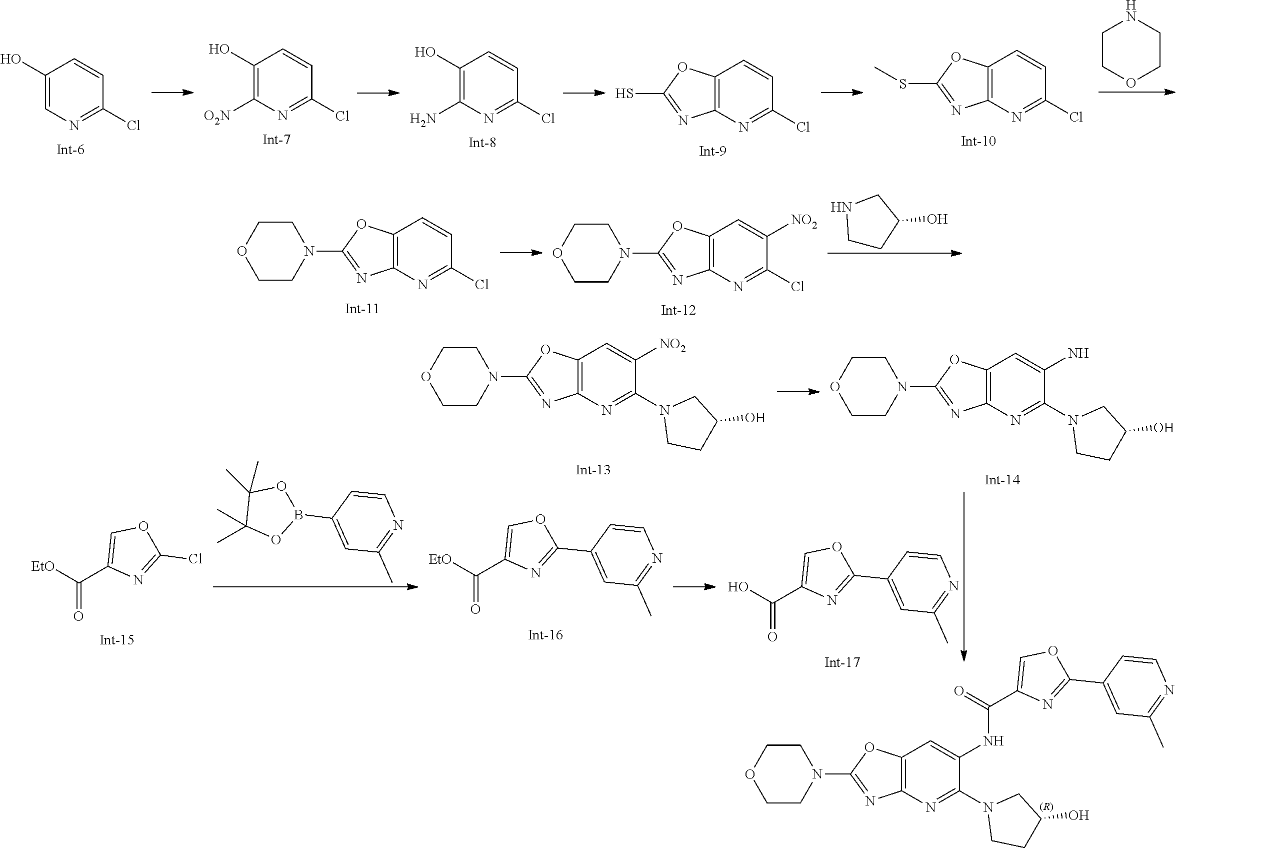

### Step 1: Preparation of 6-chloro-2-nitropyridine-3-ol (Int-7)

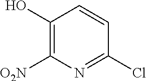 [SMILES: O=[N+]([O-])c1nc(Cl)ccc1O]

6-Chloropyridine-3-ol (11 g, 85.27 mmol) was dissolved in 50 ml of sulfuric acid, the mixture was cooled to 0° C. in an ice salt bath, and 10 ml of nitric acid (65%) solution was slowly added dropwise while maintaining the reaction temperature below 10° C. After completion of addition, the reaction solution was continued to be stirred for 3 hours, then the reaction solution was poured into 400 ml of ice water, the mixture was stirred for 30 minutes, filtered, the filter cake was washed with water for three times, and the filter cake was collected and dried to obtain 10.43 g of target compound.

MS(ESI):m/z=175.1 [M−H]⁺.

### Step 2: Preparation of 2-amino-6-chloropyridine-3-ol (Int-8)

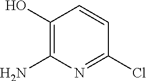

6-Chloro-2-nitropyridine-3-ol (10.43 g, 0.06 mol) was dissolved in 100 ml of ethanol, then iron filings (33.6 g, 0.6 mol) and ammonium chloride (1.6 g, 0.03 mol) were added, and the mixture was stirred at 90° C. for 3 h. The reaction solution was cooled to room temperature, filtered, the filter cake was washed three times with methanol, the filtrate was collected and concentrated, the concentrate was washed with water, and the concentrate was extracted three times with ethyl acetate. The organic phase was combined, washed with saturated brine, and dried over anhydrous sodium sulfate and concentrated to obtain 3.0 g of target compound.

MS(ESI):m/z=145.2[M+H]⁺.

### Step 3: 5-chloroxazolo [4,5-b] pyridine-2-thiol (Int-9)

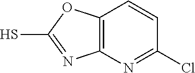 [SMILES: Sc1nc2nc(Cl)ccc2o1]

2-amino-6-chloropyridine -3-ol (3.0 g, 20.83 mmol) was dissolved in 23 ml of pyridine, then potassium ethyl xanthate (4.5 g, 28.12 mmol) was added, and the reaction solution was heated to 110° C. and stirred overnight. The reaction solution was cooled to 0° C., 200 ml of ice water was added, and acidified with concentrated hydrochloric acid. The solid was filtered, the filter cake was washed with water and dried to obtain 3.0 g of target compound.

MS(ESI):m/z=187.0[M+H]⁺.

### Step 4: Preparation of 5-chloro-2-methylthioxazolo [4,5-b] pyridine (Int-10)

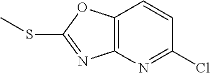

Potassium carbonate (4.5 g, 32.26 mmol) and methyl iodide (4.58 g, 32.26 mmol) were added to a solution of 5-chloroxazolo [4,5-b] pyridine-2-thiol (3.0 g, 16.13 mmol) in 50 ml ethyl acetate, and the reaction solution was stirred at room temperature for 2 h. The reaction solution was diluted with 100 ml of water and extracted with ethyl acetate for three times, the organic phase was combined, the organic phase was washed with saturated brine, and dried over anhydrous sodium sulfate and concentrated to obtain 2.75 g of target compound.

MS(ESI):m/z=200.9[M+H]⁺.

### Step 5: Preparation of 5-chloro-2-morpholinoxazolo [4,5-b] pyridine (Int-11)

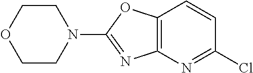 [SMILES: Clc1ccc2oc(N3CCOCC3)nc2n1]

6 ml of morpholine was added to a solution of 5-chloro-2-methylthioxazolo [4,5-b] pyridine (2.75 g, 13.71 mmol) in tetrahydrofuran(30 ml), and the reaction solution was heated to 75° C. and stirred overnight. The reaction solution was cooled to room temperature and concentrated, the concentrated solution was washed with water, extracted three times with dichloromethane, and the organic phase was combined. The organic phase was dried over anhydrous sodium sulfate and spun dry to obtain 2.62 g of target compound.

MS(ESI):m/z=240.0[M+H]⁺.

### Step 6: Preparation of 5-chloro-2-morpholin-6-nitrooxazolo [4,5-b] pyridine (Int-12)

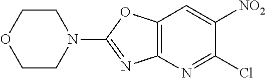 [SMILES: O=[N+]([O-])c1cc2oc(N3CCOCC3)nc2nc1Cl]

5-Chloro-2-morpholine oxazolo [4,5-b] pyridine(2.62 g) was dissolved in 25 ml of sulfuric acid, and the mixture was cooled to 0° C. in an ice salt bath. Nitric acid(1.2 ml, 65%) was slowly added dropwise to the reaction solution and the reaction temperature was maintained below 10° C. After the nitric acid was added dropwise, the reaction solution was stirred overnight at room temperature. The reaction solution was poured into 250 ml of ice water, stirred for 30 minutes, filtered, and the solid was washed with water for three times, and dried to obtain 1.86 g of target compound.

MS(ESI):m/z=284.9[M+H]⁺.

### Step 7: Preparation of (R)-1-(2-morpholin-6-nitrooxazolo [4,5-b] pyridine-5-yl) pyrrolidinyl-3-ol (Int-13)

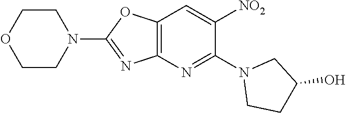 [SMILES: O=[N+]([O-])c1cc2oc(N3CCOCC3)nc2nc1N1CC[C@@H](O)C1]

5-Chloro-2-morpholin-6-nitrooxazolo [4,5-b] pyridine (1.86 g, 6.53 mmol) was dissolved in 10 ml of N,N-dimethylformamide, then (R)-pyrrolidinyl-3-ol (1.13 g, 13.05 mmol) and potassium carbonate(2.7 g, 19.6 mmol) were added, and the reaction solution was stirred overnight at room temperature. The reaction solution was diluted with water to 100 ml, extracted three times with ethyl acetate. The organic phase was combined, washed with saturated brine, dried over anhydrous sodium sulfate, and concentrated to obtain crude product which was purified by silica gel column (dichloromethane/methanol=90/10) to obtain 1.6 g of target compound.

MS(ESI):m/z=336.3[M+H]⁺;

¹H NMR (400 MHz, DMSO) δ8.22 (s, 1H), 4.96 (d, J=3.2 Hz, 1 H), 4.36 (d, J=2.0 Hz, 1 H), 3.79-3.59 (m, 9H), 3.49 (dd, J=12.1, 4.4 Hz, 1H), 3.24 (ddd, J =11.1, 7.8, 3.2 Hz, 1 H), 2.85 (d, J =12.1 Hz, 1 H), 2.02-1.81 (m, 2 H).

### Step 8: Preparation of (R)-1-(6-amino-2-morpholinoxazolo[4, 5-b]pyridine-5-yl) pyrrolidinyl-3-ol (Int-14)

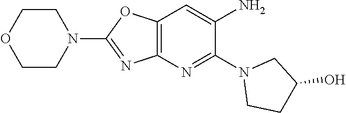 [SMILES: Nc1cc2oc(N3CCOCC3)nc2nc1N1CC[C@@H](O)C1]

(R)-1-(2-morpholin-6-nitrooxazolo [4,5-b] pyridine-5-yl) pyrrolidinyl-3-ol (300 mg, 0.895 mmol) was dissolved in 10 ml of methanol, then 3 mg 10% wet palladium/carbon was added, and the reaction solution was degassed with hydrogen three times, and stirred for hydrogenation at room temperature for 3 hours. Filtered, the solid was washed with methanol for three times, and the organic phase was collected and concentrated to obtain 250 mg of target compound.

MS(ESI):m/z=306.0[M+H]⁺.

### Step 9: Preparation of ethyl 2-(2-methylpyridine-4-yl) oxazol-4-carboxylate (Int-16)

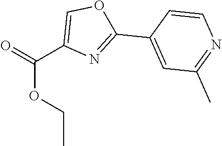 [SMILES: CCOC(=O)c1coc(-c2ccnc(C)c2)n1]

Ethyl 2-chloroxazol-4-carboxylate (2.0 g, 11.43) was dissolved in 40 ml of 1, 2-dichloroethane and 10 ml of water, then 2-methyl -4-(4,4,5,5-tetramethyl-1,3,2-dioxaboran-2-yl)pyridine (2.5 g, 11.43 mmol), Pd (dppf)C12 (0.835 g, 1.143 mmol) and potassium carbonate (3.15 g, 22.86 mmol) were added, the reaction solution was degassed under nitrogen gas for three times, then the reaction solution was heated to 80° C. for reaction overnight. The reaction solution was cooled to room temperature and diluted with water to 50 ml, extracted with dichloromethane for three times, organic phase was collected and concentrated to obtain crude product, and the crude product was purified by silica gel column (petroleum ether/ethyl acetate=60/40) to obtain 2.3 g of target compound.

MS(ESI):m/z=233.0[M+H]⁺.

### Step 10: Preparation of 2-(2-methylpyridine-4-yl) oxazol-4-carboxylic acid (Int-17)

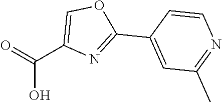

Ethyl 2-(2-methylpyridine-4-yl) oxazol-4-carboxylate (2.2 g, 9.48 mmol) was dissolved in 40 ml of ethanol and 5 ml of water, then lithium hydroxide (683 mg, 28.44) was added, and the reaction solution was stirred at room temperature overnight. The reaction solution was acidified with hydrochloric acid to a pH of about 4, and concentrated to obtain a crude product. The crude product was purified by column to obtain 1.0 g of target compound. MS(ESI):m/z=205.1[M+H]⁺.

### Step 11: Preparation of (R)-N-(5-(3-hydroxypyrrolidin-l-yl)-2-morpholinoxazo [4,5-b] pyridine-6-yl)-2-(2-methylpyridine-4-yl) oxazol-4-formamide (41)

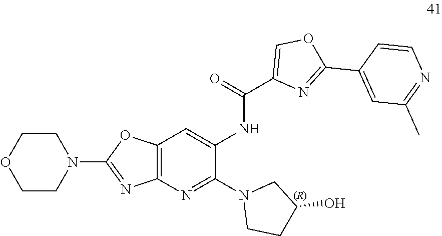

(R)-1-(6-amino-2-morpholinoxazolo [4,5-b] pyridine-5-yl) pyrrolidinyl-3-ol (250 mg, 0.820 mmol) was dissolved in 6 ml of N,N-dimethylformamide, then 2-(2-methylpyridine-4-yl) oxazol-4-carboxylic acid (201 mg, 0.984 mmol), HATU (405 mg, 1.065 mmol) and DIEA (317 mg, 2.46 mmol) were added, and then the mixture was stirred at room temperature overnight. The reaction solution was diluted with water, extracted three times with dichloromethane, the organic phase was combined and concentrated, and the concentrate was purified by silica gel column (dichloromethane/methanol =90/10) to obtain crude product which was purified by preparative HPLC to obtain 105mg of target compound.

MS(ESI):m/z=492.1[M+H]⁺;

51 H NMR (400 MHz, DMSO) δ9.81 (s, 1 H), 8.96 (s, 1 H), 8.68 (d, J=5.1 Hz, 1 H), 7.87 (s, 1 H), 7.77 (d, J=4.4 Hz, 1 H), 7.67 (s, 1 H), 4.86 (s, 1 H), 4.28 (s, 1 H), 3.79-3.69 (m, 4 H), 3.59 (dd, J=18.2, 5.3 Hz, 6H), 3.44 (dd, J=7.7, 4.2 Hz, 1 H), 3.24 (d, J=10.9 Hz, 1 H), 2.59 (s, 3 H), 1.88 (ddd, J=56.5, 32.2, 6.1 Hz, 2H).

### II BIOLOGICAL ACTIVITY TEST EXAMPLES

### Example 1. Effect of IRAK4 Inhibitor on Inflammatory Factors in Alveolar Lavage Fluid of ALI/ARDS Mice

Experimental animals: female Balb/c mice (weighing about 22-25 g), SPF grade, 36 mice, aged 6-7 weeks, purchased from Shanghai Xipuer-Bikai Laboratory Animal Co., Ltd.

Experimental method: Mice with balanced weight were randomly divided into 6 groups: normal group, model group, dexamethasone group, compound 146 group, compound 41 group and compound 450 group, and were respectively orally given vehicle, dexamethasone 10 mg/kg, compound 146 150 mg/kg, compound 41 75 mg/kg, compound 450 100 mg/kg (purchased from Abmole China), and the administration volume was 10 ml/kg. The normal group and model group were given the same volume of vehicle (5% DMSO+15% Solutol+80% PBS). After 0.5 h of administration, all animals except the normal group inhaled 3% isoflurane for anesthesia, LPS (i.e., lipopolysaccharide)(Sigma, 1750 ug/kg) was given by using a nebulizer (Aeroneb®Rev.B 30-192) via tracheal atomization to induce ALI/ARDS model. After LPS induced model for 4 hours, all mice were anesthetized and alveolar lavage fluid was collected, and the supernatant was collected by centrifuging alveolar lavage fluid. The levels of TNF-α, IL-6 and IL-1β in the supernatant were detected by enzyme-linked immunosorbent assay (Elisa). The data were represented by mean±standard error (Mean±SEM), and the differences between groups were analyzed by one-way ANOVA/Dunnett test, p<0.05 was considered to have significant differences.

The experimental results are shown in Table 1.

TABLE 1

| | IC₅₀/IRAK4 | Inhibition of | Inhibition of | Inhibition of |
|---|---|---|---|---|
| Compound | (enzymology) | TNF-α (%) | IL-6 (%) | IL-1β (%) |
| 146 | 3.4 nM | 87.30 | 82.70 | 76.20 |
| 41 | <50 nM | 12.90 | 21.40 | 52.00 |
| 450 | 0.2 nM | 28.70 | 36.40 | 43.90 |
| Normal group | | 169.90 | 36.40 | 43.90 |
| Model group | | 0.00 | −3.40 | 145.00 |
| Dexamethasone | | 128.70 | 0.00 | 0.00 |

In addition, Table 1 above also shows the IC₅₀ results of the compounds.

It can be known from above Table 1:
1) After acting for 4 hours, although compound 450 has very excellent IC₅₀ results than compound 146, its inhibition rate on TNF-α, IL-6 and IL-1β factors is significantly lower than that of compound 146; in other words, compared with the same IRAK4 inhibitor compound 450, IRAK4 inhibitor compound 146 has significantly excellent TNF-α, IL-6 and IL-1β inhibitory effects.
2) After acting for 4 hours, IRAK4 inhibitor compound 146 has significantly excellent TNF-α, IL-6 and IL-1β inhibitory effects than IRAK4 inhibitor compound 41.
3) After acting for 4 hours, compound 146 has significantly excellent inhibitory effects on TNF-α, IL-6 and IL-10 compared with model group.

### Example 2. Effect of IRAK4 Inhibitor on Cell Count Level of Alveolar Lavage Fluid and Lung Pathological Changes in ALI/ARDS Mice

Experimental animals: female Balb/c mice, SPF grade, 36 mice, aged 6-7 weeks, purchased from Shanghai Xipuer-Bikai Laboratory Animal Co., Ltd. Experimental method: The mice with balanced weight were randomly divided into 6 groups: normal group, model group, dexamethasone group, compound 146 group, compound 41 group and compound 450 group, and were respectively orally given vehicle, dexamethasone 10 mg/kg, compound 146 150 mg/kg, compound 41 75 mg/kg, compound 450 100 mg/kg (purchased from abmole China), and the administration volume was 10 ml/kg, the normal group and the model group were given the same volume of vehicle (5% DMSO+15% Solutol+80% PBS). After 0.5 h of administration, all animals except the normal group inhaled 3% isoflurane for anesthesia, LPS(Sigma, 1750 ug/kg) was given by using a nebulizer (Aeroneb®Rev.B 30-192) via tracheal atomization to induce ALI/ARDS model. LPS induced model was given again after 6 hours, and the dose was the same as that of the first administration. After 24 hours from LPS induced model, all mice were weighed, the animals used were anesthetized and the alveolar lavage fluid was collected, the lower cell pellets were collected after centrifugation, and the cells were resuspended, fixed with methanol and stained with Wright-Giemsa, and the eosinophils, neutrophils, macrophages and lymphocytes were counted by an optical microscope. The mice were euthanized with excess carbon dioxide. After the maximum blood volume was collected from the heart, the left lung was fixed with neutral formaldehyde and embedded in paraffin, sliced and HE staining, photographed to observe the lung injury and score. The data were represented by mean±standard error (Mean±SEM), the differences between groups were analyzed by one-way ANOVA/Dunnett test, and p<0.05 was considered to have significant differences.

Lung pathology scoring criteria: \textcircled{1} alveolar congestion; \textcircled{2} hemorrhage; \textcircled{3} neutrophil infiltration into airspace or vascular wall; \textcircled{4} alveolar wall/thickness formed by hyaline membrane. Each category was scored on a scale of 0 to 4: 0=no damage, 1=damage field ratio<25%, 2=25% <damage field ratio≤50%, 3=50% <damage field ratio≤75%, 4=diffuse damage, and pathological score was completed by professional and technical personnel using blind method. Experimental results: Table 2 shows the results after 24 hours of acting.

TABLE 2

| | | Total cell | | | | | Pulmonary |
|---|---|---|---|---|---|---|---|
| | | count | Eosinophils | Macrophages | Neutrophils | Lymphocytes | pathology |
| Compound | Weight (g) | (*10⁴) | (*10⁴) | (*10⁴) | (*10⁴) | (*10⁴) | score |
| 146 | 20.66 ± 0.78 | 265.13 | 1.83 | 86.29 | 175.40 | 1.61 | 2.83 |
| 41 | 19.45 ± 0.71 | 561.75 | 2.81 | 89.06 | 466.17 | 3.71 | 4.17 |
| 450 | 18.68 ± 0.4 | 467.50 | 3.11 | 74.91 | 386.74 | 2.74 | 4.83 |
| Normal group | 19.7 ± 0.57 | 249.13 | 2.70 | 205.47 | 36.46 | 4.50 | 4.17 |
| Model group | 18.67 ± 0.28 | 986.38 | 5.61 | 77.56 | 892.51 | 10.69 | 5.00 |
| Dexamethasone | 18.91 ± 0.74 | 476.25 | 3.17 | 108.20 | 361.63 | 3.25 | 3.50 |

It can be known from above Table 2:
1) Compared with compound 41 and compound 450, compound 146 can effectively maintain the weight of mice
2) Compared with normal group, model group and dexamethasone group, compound 146 can also effectively maintain the weight of mice.
3) Compared with compound 41 and compound 450, compound 146 has significantly reduced total cell count, eosinophil count, neutrophil count, and lymphocyte count.
4) Compared with the model group, compound 146 has significantly reduced the total cell count, eosinophil count, neutrophil count and lymphocyte count.
5) Compared with compound 41, compound 450, normal group, model group and dexamethasone group, compound 146 has significantly improved lung pathological score

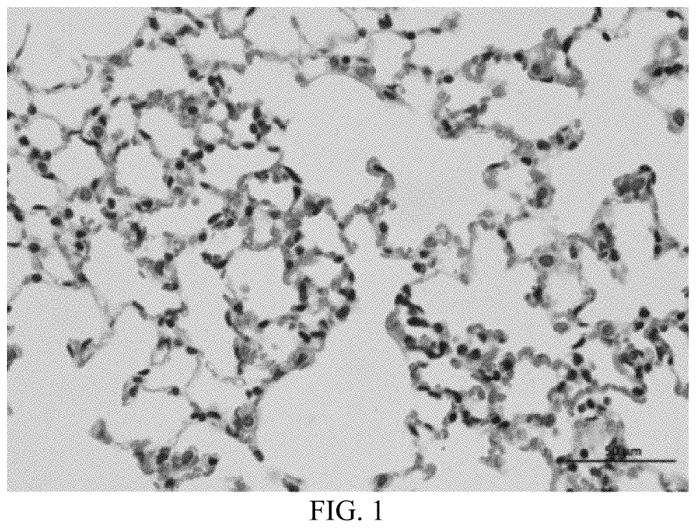

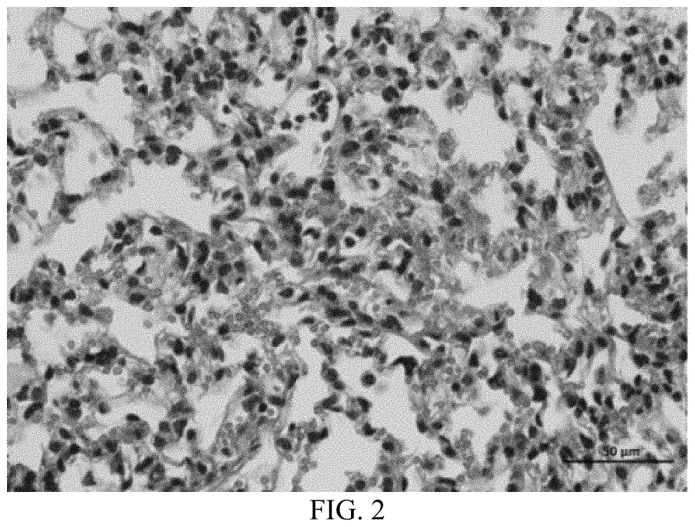

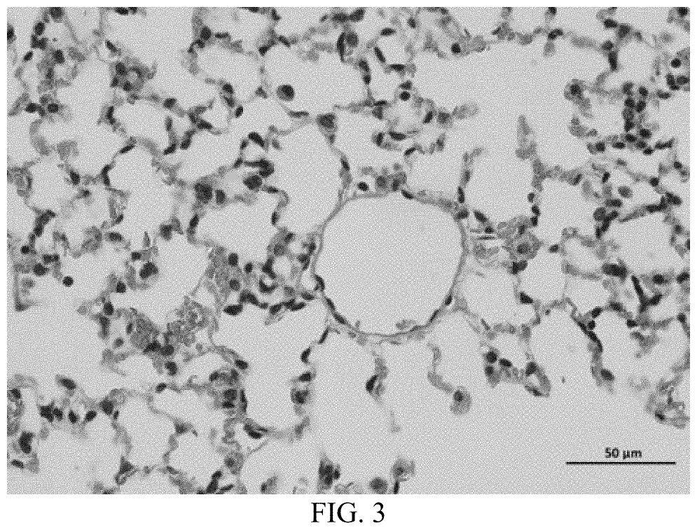

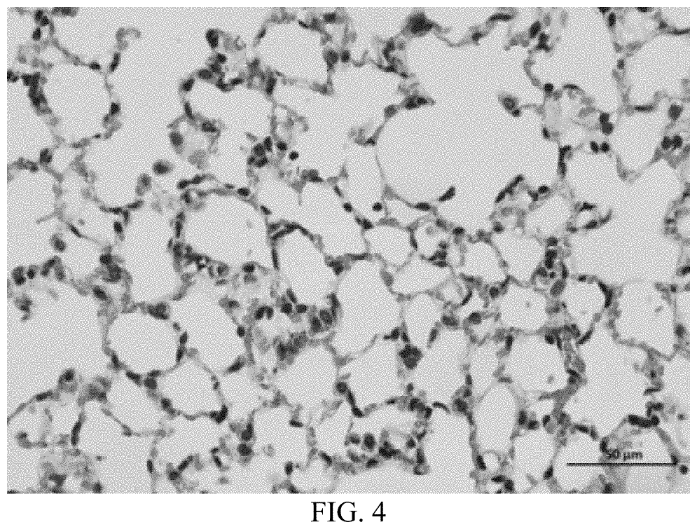

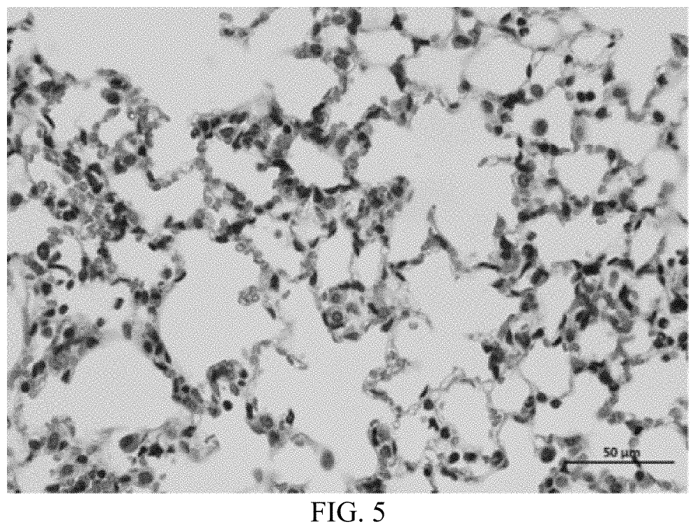

Combined with FIGS. 1-5, the results showed that compared with LPS-induced ALI/ARDS model group, the score of lung pathological damage in mice after compound 146 treatment was significantly reduced, indicating that compound 146 also significantly improved pathological changes such as congestion, inflammatory infiltration and alveolar wall thickening/hyaline membrane formation in model mice.

1. Use of an IRAK4 inhibitor for the preparation of a drug,
wherein the IRAK4 inhibitor is a compound of formula I; 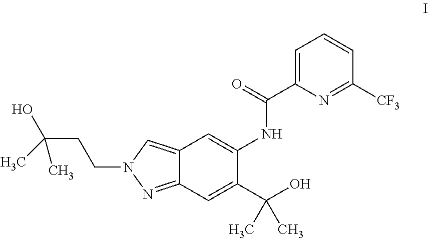 [SMILES: CC(C)(O)CCn1cc2cc(NC(=O)c3cccc(C(F)(F)F)n3)c(C(C)(C)O)cc2n1]
the drug is used to prevent and/or treat a disease selected from the group consisting of:
1) acute lung injury;
2) acute respiratory distress syndrome; and
3) acute lung injury or acute respiratory distress syndrome related disease.

2. The use of claim 1, wherein the acute lung injury or the acute respiratory distress syndrome is caused by direct lung injury or indirect lung injury;
the direct lung injury is lung injury caused by lung contusion, aspiration, drowning, poison inhalation, diffuse lung infection, bacteria, virus, toxin or hypoxia;
the indirect lung injury is severe injury, shock, severe infection, severe non-chest trauma, sepsis, acute pancreatitis, massive blood transfusion, extracorporeal circulation, diffuse intravascular coagulation, or dermatomyositis.

3. The use of claim 1, wherein the acute lung injury or the acute respiratory distress syndrome related disease is selected from the group consisting of non-cardiogenic pulmonary edema, acute hypoxic respiratory insufficiency or failure, hypoxemia, pulmonary interstitial fibrosis, pulmonary hypertension, and a combination thereof.

4. The use of claim 1, wherein the disease is mediated by a cytokine selected from the group consisting of TNF-α, IL-6, IL-1β, and a combination thereof

5. The use of claim 1, wherein the disease is mediated by inflammatory cells selected from the group consisting of eosinophils, neutrophils, lymphocytes, and combinations thereof.

6. The use of claim 1, wherein the disease is an LPS-induced disease.

7. The use of claim 6, wherein the drug is administered before LPS induction.

8. The use of claim 7, wherein the drug is administered again after LPS induction.

9. The use of claim 1, wherein the drug comprises:
1) a prophylactic and/or therapeutically effective amount of the compound of formula I; and
2) pharmaceutically acceptable carriers, diluents or excipients.

10. A drug for the prevention and/or treatment of acute lung injury or acute respiratory distress syndrome or related disease thereof, wherein the drug comprises:
1) the compound of formula I as the first active ingredient; 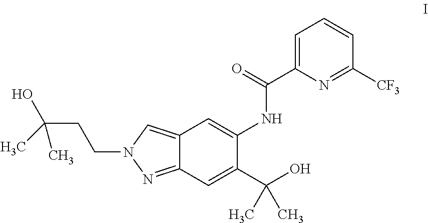 [SMILES: CC(C)(O)CCn1cc2cc(NC(=O)c3cccc(C(F)(F)F)n3)c(C(C)(C)O)cc2n1]
2) an optional second active ingredient, the second active ingredient is different from the first active ingredient, and the second active ingredient is used to prevent and/or treat acute lung injury or acute respiratory distress syndrome or related disease thereof;
3) pharmaceutically acceptable carriers, diluents or excipients.
